(19) United States
(12) Patent Application Publication
(10) Pub. No.: US 2010/0008966 A1
(43) Pub. Date: Jan. 14, 2010

(54) Medical Devices and Methods for Delivery of Nucleic Acids

(75) Inventors: Joram Slager, St. Louis Park, MN (US); Joseph Schmidt McGonigle, Minneapolis, MN (US); Aron Brent Anderson, Minnetonka, MN (US); Robert W. Hergenrother, Eden Prairie, MN (US)

Correspondence Address: PAULY, DEVRIES SMITH & DEFFNER, L.L.C., Plaza VII-Suite 3000, 45 South Seventh Street, MINNEAPOLIS, MN, 55402-1630, US

(73) Assignee: SURMODICS, INC., Eden Prairie, MN (US)

(21) Appl. No.: 12/502,473

(22) Filed: Jul. 14, 2009

Related U.S. Application Data
(60) Provisional application No. 61/080,483, filed on Jul. 14, 2008.

(51) Int. Cl. A61K 9/14; A61K 31/7088; A61K 9/00; A61K 31/7105; A61P 35/00

(52) U.S. Cl. 424/422; 424/486; 514/44 R; 514/44 A

(57) ABSTRACT

Embodiments of the invention include devices for the release of nucleic acids and related methods. In an embodiment, the invention includes an active agent eluting coating including a polymeric matrix, a cationic carrier agent disposed with the matrix, and an active agent disposed within the matrix, the active agent including nucleic acids substantially uncomplexed with the cationic carrier agent. In an embodiment, the invention includes a method of making an implantable medical device including selecting a concentration of a cationic carrier agent corresponding to a desired elution profile, combining a matrix forming polymer, an active agent, a solvent, and the cationic carrier agent to form a coating composition having the selected concentration of the cationic carrier agent, the active agent comprising nucleic acids, and depositing the coating composition onto the surface of a substrate. Other embodiments are included herein.

This application claims the benefit of U.S. Provisional Application No. 61/080,483, filed Jul. 14, 2008, the contents of which are herein incorporated by reference.

### FIELD OF THE INVENTION

The present invention relates to devices and methods for the release of active agents. More specifically, the present invention relates to devices and methods for the release of nucleic acids.

### BACKGROUND OF THE INVENTION

One promising approach to the treatment of various medical conditions is the administration of nucleic acids as therapeutic agents. By way of example, this approach can include the administration of RNA, DNA, siRNA, miRNA, piRNA, shRNA, antisense nucleic acids, aptamers, ribozymes, catalytic DNA and the like.

However, successful treatment with nucleic acids can depend on many factors. Specifically, in order to mediate an effect on a target cell, a nucleic acid based active agent must generally be delivered to an appropriate target cell, taken up by the cell, released from an endosome, and transported to the nucleus or cytoplasm (intracellular trafficking), among other steps. As such, successful treatment with nucleic acids depends upon site-specific delivery, stability during the delivery phase, and a substantial degree of biological activity within target cells. For various reasons, these steps can be difficult to achieve.

One technique for administering nucleic acid based active agents is to use an implantable medical device as a delivery platform. The use of implantable medical devices for this purpose can provide site specific delivery of nucleic acids. However, there are various practical challenges associated with the use of such medical devices including manufacturing challenges, shelf stability, desirable elution profiles, sufficient active agent loading, and the like.

Accordingly, a need still remains for devices that can deliver therapeutic nucleic acids to a target tissue and methods of making and using the same.

### SUMMARY OF THE INVENTION

Embodiments of the invention include devices for the release of nucleic acids and related methods. In an embodiment, the invention includes an active agent eluting coating including a polymeric matrix, a cationic carrier agent disposed with the matrix, and an active agent disposed within the matrix, the active agent including nucleic acids substantially uncomplexed with the cationic carrier agent.

In an embodiment, the invention includes an implantable medical device including a substrate, and a coating disposed on the substrate. The coating can include a polymeric matrix, a cationic carrier agent disposed with the matrix, and an active agent disposed within the matrix, the active agent including nucleic acids substantially uncomplexed with the cationic carrier agent.

In an embodiment, the invention includes a method of making an implantable medical device. The method can include selecting a concentration of a cationic carrier agent corresponding to a desired elution profile. The method can also include combining a matrix forming polymer, an active agent, a solvent, and the cationic carrier agent to form a coating composition having the selected concentration of the cationic carrier agent, the active agent comprising nucleic acids. The method can further include depositing the coating composition onto the surface of a substrate.

In an embodiment, the invention includes a liquid composition for forming an active agent eluting coating. The liquid composition can include a matrix forming polymer, a cationic carrier agent, an active agent, the active agent including nucleic acids; and a solvent, wherein the active agent is not soluble in the solvent.

In an embodiment, the invention includes a method of making an implantable medical device. The method can include combining a matrix forming polymer mixture, a cationic carrier agent, and an active agent together with a non-aqueous solvent to form a monophasic coating composition, the active agent comprising nucleic acids. The method can also include depositing the coating solution onto the surface of a substrate.

In an embodiment, the invention can include a liquid composition for forming an active agent eluting coating. The liquid composition can include a matrix forming polymer, a cationic carrier agent, and an active agent. The active agent can include nucleic acids. The liquid composition can also include a solvent, wherein the active agent is not soluble in the solvent.

The above summary of the present invention is not intended to describe each discussed embodiment of the present invention. This is the purpose of the figures and the detailed description that follows.

### BRIEF DESCRIPTION OF THE FIGURES

The invention may be more completely understood in connection with the following drawings, in which:

FIG. 1 is a graph of siRNA release from a coating over time.

FIG. 2 is a graph of siRNA release from a coating over time as affected by varying amounts of polyethyleneimine (PEI).

FIG. 3 is a graph of siRNA release from a coating over time as affected by varying amounts of PEI.

FIG. 4 is a graph of DNA release from a coating over time.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

As used herein, the term “complex” shall refer to a chemical association of two or more chemical species through non-covalent bonds. The term “uncomplexed” in reference to two or more chemical species in a mixture shall refer to the property of those chemical species not being associated to one another through covalent or non-covalent chemical bonds. The term “substantially uncomplexed” in reference to two or more chemical species in a mixture shall refer to the property of the species existing substantially independently with only negligible complexation taking place.

Nucleic acids, such as RNA and DNA, are generally only soluble in polar solvents, such as aqueous solvents, having limited or no solubility in non-polar solvents. In many cases non-polar solvents can actually be harmful to the activity of nucleic acids. However, there are techniques that can be used to prepare nucleic acids for contact with non-polar solvents. For example, there are various techniques, such as lyophilization, to prepare nucleic acids as a particulate, allowing the nucleic acids to maintain sufficient biological activity while in contact with non-polar solvents.

Cationic carrier agents can be useful for the delivery of nucleic acid based active agents. Cationic carrier agents can form a complex with nucleic acids. The resulting complex can be useful for maintaining activity of the nucleic acids in aqueous environments, in addition to aiding transfection. However, the formation of complexes between cationic carrier agents and nucleic acids generally requires the presence of aqueous solvents. As such, complex formation does not take place solely in the presence of non-aqueous solvents.

Applicants have developed methods of creating medical devices that include both nucleic acids and cationic carrier agents, but do not include complexes between the two until after the device is inserted in vivo. For example, in an embodiment, the invention includes a method of making an implantable medical device including combining a matrix forming polymer mixture, a cationic carrier agent, and nucleic acids together with a non-aqueous solvent to form a coating composition and then depositing the coating solution onto the surface of a substrate. Because the coating composition does not include an aqueous solvent, complexes do not form between the nucleic acids and the cationic carrier agent.

While not intending to be bound by theory, it is believed that when devices including both nucleic acids and cationic carrier agents, but not including complexes of the two, are inserted into an aqueous environment, such as that present in vivo, water can permeate the coating and cause the formation of complexes between the nucleic acids and the cationic carrier agents. As such, during manufacturing and shelf storage, substantially no complexes exist in the coating of the device. However, after such medical devices are implanted into a subject, or otherwise exposed to an environment including aqueous solvents, the presence of water can cause the cationic carrier agent to form complexes with the nucleic acids and then elute out of the coating. As such, in accordance with various embodiments herein, the formation of complexes between cationic carrier agents and nucleic acids can be delayed until the time of actual device use.

Delayed formation of complexes between cationic carrier agents and nucleic acids can offer various advantages. For example, it is believed that such devices exhibit enhanced shelf stability over otherwise similar devices where nucleic acids are already complexed to cationic carrier agents. Delayed complex formation is expected to reduce aggregation and loss of activity typically seen in aqueous solution with nucleic acid/cationic carrier complexes. In addition, nucleic acids are expected to be more stable when maintained in organic phase as they are not readily accessible by degradative enzymes such as nucleases.

Maintaining nucleic acids in particulate form suspended in an organic solvent during the device manufacturing process can also offer various practical advantages. For example, one desirable technique for applying a liquid composition onto a substrate in order to form a coating is spray coating. Spray coating can be desirable because it can be used to deposit coatings with a level of precision that is difficult to achieve with other techniques such as dip coating. However, in order for spray coating to work, the liquid composition to be sprayed must have certain properties, such as a viscosity less than a threshold amount and a maximum particle size less than a threshold amount, that make it conducive to spray application. Some liquid compositions simply cannot be spray-coated onto a substrate and result in desirable coatings. It has been found, however, that maintaining nucleic acids in particulate form in a liquid composition prior to and during the application process can result in desirable coatings.

In some embodiments herein, matrix forming polymers used include both degradable and non-degradable polymers. While not intending to be bound by theory, the use of both degradable and non-degradable polymers can offer various advantages. For example, some coatings that only include non-degradable polymers may not have desirable elution properties. Coatings that only include degradable polymers may be more likely to shed pieces of the coating into the in vivo environment which may be undesirable in some applications. Specific aspects of exemplary embodiments will now be described in greater detail.

### Cationic Carrier Agents

Exemplary classes of suitable cationic carrier agents can include cationic polymers and cationic lipids. Suitable cationic carrier agents can also include polycation containing cyclodextrin, histones, cationized human serum albumin, aminopolysaccharides such as chitosan, peptides such as poly-L-lysine, poly-L-ornithine, and poly(4-hydroxy-L-proline ester, and polyamines such as polyethylenimine (PEI), polypropylenimine, polyamidoamine dendrimers, and poly(beta-aminoesters). Other carrier agents can include liposomes, protein transduction domains and polyvinyl pyrrolidone (PVP). Additionally, carriers may also be conjugated to molecules which allow them to target specific cell types. Examples of targeting agents include antibodies and peptides which recognize and bind to specific cell surface molecules.

### Active Agents

Nucleic acids used with embodiments of the invention can include various types of nucleic acids that can function to provide a therapeutic effect. Exemplary types of nucleic acids can include, but are not limited to, ribonucleic acids (RNA), deoxyribonucleic acids (DNA), small interfering RNA (siRNA), micro RNA (miRNA), piwi-interacting RNA (piRNA), short hairpin RNA (shRNA), antisense nucleic acids, aptamers, ribozymes, locked nucleic acids and catalytic DNA.

In various embodiments, nucleic acids used with embodiments of the invention can include derivatives of the above. Derivatives can include chemically modified nucleic acids and nucleic acids with components such as lipids or polymer conjugated thereto. By way of example, chemical modifications can include altered chemistry of individual nucleotides within the nucleic acids. Specific examples of chemical modification can include phosphodiester modifications such as phosphorothioate RNA and boranophosphonate RNA as well as 2′ sugar modifications such as 2′-O-methyl RNA, 2′-deoxy-2′-fluoro RNA and locked nucleic acids. Chemical modification can also include methylation, modification with a halogen such as fluorination, and the like.

Direct conjugation of lipids and polymers to siRNA can facilitate intracellular delivery and gene silencing in the absence of complexed carrier agents. An example is the conjugation of cholesterol to the 3′ sense strand of siRNA which results in knockdown in vivo after intravenous administration (Soutschek et al. 2004, Therapeutic silencing of an endogenous gene by systemic administration of modified siRNAs, Nature 432: 173-178). Additionally, siRNA can be directly conjugated to peptides, lipids and other molecules which can lead to cellular uptake and functional knockdown (see De Paula et al., Hydrophobization and bioconjugation for enhanced siRNA delivery and targeting, RNA 2007 13: 431-456).

### Matrix Forming Polymers

Matrix forming polymers used with embodiments of the invention can include degradable polymers and/or non-degradable polymers.

Degradable polymers used with embodiments of the invention can include both natural or synthetic polymers. Examples of degradable polymers can include those with hydrolytically unstable linkages in the polymeric backbone. Degradable polymers can include degradable block copolymers including amphiphilic blocks. Degradable polymers of the invention can include both those with bulk erosion characteristics and those with surface erosion characteristics.

Synthetic degradable polymers can include: degradable polyesters (such as poly(glycolic acid), poly(lactic acid), poly(lactic-co-glycolic acid), poly(dioxanone), polylactones (e.g., poly(caprolactone)), poly(3-hydroxybutyrate), poly(3-hydroxyvalerate), poly(valerolactone), poly(tartronic acid), poly(β-malonic acid), poly(propylene fumarate)); degradable polyesteramides; degradable polyanhydrides (such as poly(sebacic acid), poly(1,6-bis(carboxyphenoxy)hexane, poly(1,3-bis(carboxyphenoxy)propane); degradable polycarbonates (such as tyrosine-based polycarbonates); degradable polyiminocarbonates; degradable polyarylates (such as tyrosine-based polyarylates); degradable polyorthoesters; degradable polyurethanes; degradable polyphosphazenes; and copolymers thereof.

Natural or naturally-based degradable polymers can include polysaccharides and modified polysaccharides such as starch, cellulose, chitin, chitosan, and copolymers thereof.

Specific examples of degradable polymers include poly(ether ester) multiblock copolymers based on poly(ethylene glycol) (PEG) and poly(butylene terephthalate) that can be described by the following general structure:

[—(OCH₂CH₂)ₙ—O—C(O)—C₆H₄—C(O)-]x[-O—(CH₂)₄—O—C(O)—C₆H₄—C(O)-]y,

where —C₆H₄— designates the divalent aromatic ring residue from each esterified molecule of terephthalic acid, n represents the number of ethylene oxide units in each hydrophilic PEG block, x represents the number of hydrophilic blocks in the copolymer, and y represents the number of hydrophobic blocks in the copolymer. The subscript “n” can be selected such that the molecular weight of the PEG block is between about 300 and about 4000. The block copolymer can be engineered to provide a wide array of physical characteristics (e.g., hydrophilicity, adherence, strength, malleability, degradability, durability, flexibility) and active agent release characteristics (e.g., through controlled polymer degradation and swelling) by varying the values of n, x and y in the copolymer structure. Such degradable polymers can specifically include those described in U.S. Pat. No. 5,980,948, the content of which is herein incorporated by reference in its entirety.

Degradable polyesteramides can include those formed from the monomers OH-x-OH, z, and COOH-y-COOH, wherein x is alkyl, y is alkyl, and z is leucine or phenylalanine. Such degradable polyesteramides can specifically include those described in U.S. Pat. No. 6,703,040, the content of which is herein incorporated by reference in its entirety.

Degradable polymeric materials can also be selected from: (a) non-peptide polyamino polymers; (b) polyiminocarbonates; (c) amino acid-derived polycarbonates and polyarylates; and (d) poly(alkylene oxide) polymers.

In an embodiment, the degradable polymeric material is composed of a non-peptide polyamino acid polymer. Exemplary non-peptide polyamino acid polymers are described, for example, in U.S. Pat. No. 4,638,045 (“Non-Peptide Polyamino Acid Bioerodible Polymers,” Jan. 20, 1987). Generally speaking, these polymeric materials are derived from monomers, including two or three amino acid units having one of the following two structures illustrated below:

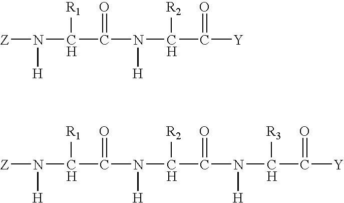

wherein the monomer units are joined via hydrolytically labile bonds at not less than one of the side groups R₁, R₂, and R₃, and where R₁, R₂, R₃ are the side chains of naturally occurring amino acids; Z is any desirable amine protecting group or hydrogen; and Y is any desirable carboxyl protecting group or hydroxyl. Each monomer unit comprises naturally occurring amino acids that are then polymerized as monomer units via linkages other than by the amide or “peptide” bond. The monomer units can be composed of two or three amino acids united through a peptide bond and thus comprise dipeptides or tripeptides. Regardless of the precise composition of the monomer unit, all are polymerized by hydrolytically labile bonds via their respective side chains rather than via the amino and carboxyl groups forming the amide bond typical of polypeptide chains. Such polymer compositions are nontoxic, are degradable, and can provide zero-order release kinetics for the delivery of active agents in a variety of therapeutic applications. According to these aspects, the amino acids are selected from naturally occurring L-alpha amino acids, including alanine, valine, leucine, isoleucine, proline, serine, threonine, aspartic acid, glutamic acid, asparagine, glutamine, lysine, hydroxylysine, arginine, hydroxyproline, methionine, cysteine, cystine, phenylalanine, tyrosine, tryptophan, histidine, citrulline, ornithine, lanthionine, hypoglycin A, β-alanine, γ-amino butyric acid, α aminoadipic acid, canavanine, venkolic acid, thiolhistidine, ergothionine, dihydroxyphenylalanine, and other amino acids well recognized and characterized in protein chemistry.

Degradable polymers of the invention can also include polymerized polysaccharides such as those described in U.S. Publ. Pat. Application No. 2005/0255142, entitled “COATINGS FOR MEDICAL ARTICLES INCLUDING NATURAL BIODEGRADABLE POLYSACCHARIDES”, U.S. Publ. Pat. Application No. 2007/0065481, entitled “COATINGS INCLUDING NATURAL BIODEGRADABLE POLYSACCHARIDES AND USES THEREOF”, and in U.S. Publ. Pat. Application No. 20070218102, entitled “HYDROPHOBIC DERIVATIVES OF NATURAL BIODEGRADABLE POLYSACCHARIDES”, all of which are herein incorporated by reference in their entirety.

Degradable polymers of the invention can also include dextran based polymers such as those described in U.S. Pat. No. 6,303,148, entitled “PROCESS FOR THE PREPARATION OF A CONTROLLED RELEASE SYSTEM”, the content of which is herein incorporated by reference in its entirety. Exemplary dextran based degradable polymers including those available commercially under the trade name OCTODEX.

Degradable polymers of the invention can further include collagen/hyaluronic acid polymers.

Degradable polymers of the invention can include multi-block copolymers, comprising at least two hydrolysable segments derived from pre-polymers A and B, which segments are linked by a multi-functional chain-extender and are chosen from the pre-polymers A and B, and triblock copolymers ABA and BAB, wherein the multi-block copolymer is amorphous and has one or more glass transition temperatures (Tg) of at most 37° C. (Tg) at physiological (body) conditions. The pre-polymers A and B can be a hydrolysable polyester, polyetherester, polycarbonate, polyestercarbonate, polyanhydride or copolymers thereof, derived from cyclic monomers such as lactide (L,D or L/D), glycolide, ε-caprolactone, δ-valerolactone, trimethylene carbonate, tetramethylene carbonate, 1,5-dioxepane-2-one, 1,4-dioxane-2-one (para-dioxanone) or cyclic anhydrides (oxepane-2,7-dione). The composition of the pre-polymers may be chosen in such a way that the maximum glass transition temperature of the resulting copolymer is below 37° C. at body conditions. To fulfill the requirement of a Tg below 37° C., some of the above-mentioned monomers or combinations of monomers may be more preferred than others. This may by itself lower the Tg, or the pre-polymer is modified with a polyethylene glycol with sufficient molecular weight to lower the glass transition temperature of the copolymer. The degradable multi-block copolymers can include hydrolysable sequences being amorphous and the segments may be linked by a multifunctional chain-extender, the segments having different physical and degradation characteristics. For example, a multi-block co-polyester consisting of a glycolide-ε-caprolactone segment and a lactide-glycolide segment can be composed of two different polyester pre-polymers. By controlling the segment monomer composition, segment ratio and length, a variety of polymers with properties that can easily be tuned can be obtained. Such degradable multi-block copolymers can specifically include those described in U.S. Publ. App. No. 2007/0155906, the content of which is herein incorporated by reference in its entirety.

Non-degradable polymer used with embodiments of the invention can include both natural or synthetic polymers. In an embodiment, the non-degradable polymer includes a plurality of polymers, including a first polymer and a second polymer. When the coating solution contains only one polymer, it can be either a first or second polymer as described herein. As used herein, the term “(meth)acrylate”, when used in describing polymers, shall mean the form including the methyl group (methacrylate) or the form without the methyl group (acrylate).

First polymers of the invention can include a polymer selected from the group consisting of poly(alkyl(meth)acrylates) and poly(aromatic(meth)acrylates), where “(meth)” will be understood by those skilled in the art to include such molecules in either the acrylic and/or methacrylic form (corresponding to the acrylates and/or methacrylates, respectively). An exemplary first polymer is poly(n-butyl methacrylate) (pBMA). Such polymers are available commercially, e.g., from Aldrich, with molecular weights ranging from about 200,000 Daltons to about 320,000 Daltons, and with varying inherent viscosity, solubility, and form (e.g., as crystals or powder). In some embodiments, poly(n-butyl methacrylate) (PBMA) is used with a molecular weight of about 200,000 Daltons to about 300,000 Daltons.

Examples of suitable first polymers also include polymers selected from the group consisting of poly(aryl(meth)acrylates), poly(aralkyl(meth)acrylates), and poly(aryloxyalkyl(meth)acrylates). Such terms are used to describe polymeric structures wherein at least one carbon chain and at least one aromatic ring are combined with acrylic groups, typically esters, to provide a composition. In particular, exemplary polymeric structures include those with aryl groups having from 6 to 16 carbon atoms and with weight average molecular weights from about 50 to about 900 kilodaltons. Suitable poly(aralkyl(meth)acrylates), poly(arylalky(meth)acrylates) or poly(aryloxyalkyl (meth)acrylates) can be made from aromatic esters derived from alcohols also containing aromatic moieties. Examples of poly(aryl(meth)acrylates) include poly(9-anthracenyl methacrylate), poly(chlorophenylacrylate), poly(methacryloxy-2-hydroxybenzophenone), poly(methacryloxybenzotriazole), poly(naphthylacrylate) and -methacrylate), poly(4-nitrophenyl acrylate), poly(pentachloro(bromo, fluoro) acrylate) and -methacrylate), and poly(phenyl acrylate) and -methacrylate). Examples of poly(aralkyl(meth)acrylates) include poly(benzyl acrylate) and -methacrylate), poly(2-phenethyl acrylate) and -methacrylate, and poly(1-pyrenylmethyl methacrylate). Examples of poly(aryloxyalkyl (meth)acrylates) include poly(phenoxyethyl acrylate) and -methacrylate), and poly(polyethylene glycol phenyl ether acrylates) and -methacrylates with varying polyethylene glycol molecular weights.

Examples of suitable second polymers are available commercially and include poly(ethylene-co-vinyl acetate) (pEVA) having vinyl acetate concentrations of between about 10% and about 50% (12%, 14%, 18%, 25%, 33% versions are commercially available), in the form of beads, pellets, granules, etc. The pEVA co-polymers with lower percent vinyl acetate become increasingly insoluble in typical solvents, whereas those with higher percent vinyl acetate become decreasingly durable.

An exemplary polymer mixture includes mixtures of pBMA and pEVA. This mixture of polymers can be used with absolute polymer concentrations (i.e., the total combined concentrations of both polymers in the coating material), of between about 0.25 wt. % and about 99 wt. %. This mixture can also be used with individual polymer concentrations in the coating solution of between about 0.05 wt. % and about 99 wt. %.

In one embodiment the polymer mixture includes pBMA with a molecular weight of from 100 kilodaltons to 900 kilodaltons and a pEVA copolymer with a vinyl acetate content of from 24 to 36 weight percent. In an embodiment the polymer mixture includes pBMA with a molecular weight of from 200 kilodaltons to 300 kilodaltons and a pEVA copolymer with a vinyl acetate content of from 24 to 36 weight percent. The concentration of the active agent or agents dissolved or suspended in the coating mixture can range from 0.01 to 99 percent, by weight, based on the weight of the final coating material.

Second polymers can also comprise one or more polymers selected from the group consisting of (i) poly(alkylene-co-alkyl(meth)acrylates, (ii) ethylene copolymers with other alkylenes, (iii) polybutenes, (iv) diolefin derived non-aromatic polymers and copolymers, (v) aromatic group-containing copolymers, and (vi) epichlorohydrin-containing polymers.

Poly(alkylene-co-alkyl(meth)acrylates) include those copolymers in which the alkyl groups are either linear or branched, and substituted or unsubstituted with non-interfering groups or atoms. Such alkyl groups can comprise from 1 to 8 carbon atoms, inclusive. Such alkyl groups can comprise from 1 to 4 carbon atoms, inclusive. In an embodiment, the alkyl group is methyl. In some embodiments, copolymers that include such alkyl groups can comprise from about 15% to about 80% (wt) of alkyl acrylate. When the alkyl group is methyl, the polymer contains from about 20% to about 40% methyl acrylate in some embodiments, and from about 25% to about 30% methyl acrylate in a particular embodiment. When the alkyl group is ethyl, the polymer contains from about 15% to about 40% ethyl acrylate in an embodiment, and when the alkyl group is butyl, the polymer contains from about 20% to about 40% butyl acrylate in an embodiment.

Alternatively, second polymers can comprise ethylene copolymers with other alkylenes, which in turn, can include straight and branched alkylenes, as well as substituted or unsubstituted alkylenes. Examples include copolymers prepared from alkylenes that comprise from 3 to 8 branched or linear carbon atoms, inclusive. In an embodiment, copolymers prepared from alkylene groups that comprise from 3 to 4 branched or linear carbon atoms, inclusive. In a particular embodiment, copolymers prepared from alkylene groups containing 3 carbon atoms (e.g., propene). By way of example, the other alkylene is a straight chain alkylene (e.g., 1-alkylene). Exemplary copolymers of this type can comprise from about 20% to about 90% (based on moles) of ethylene. In an embodiment, copolymers of this type comprise from about 35% to about 80% (mole) of ethylene. Such copolymers will have a molecular weight of between about 30 kilodaltons to about 500 kilodaltons. Exemplary copolymers are selected from the group consisting of poly(ethylene-co-propylene), poly(ethylene-co-1-butene), poly(ethylene-co-1-butene-co-1-hexene) and/or poly(ethylene-co-1-octene).

“Polybutenes” include polymers derived by homopolymerizing or randomly interpolymerizing isobutylene, 1-butene and/or 2-butene. The polybutene can be a homopolymer of any of the isomers or it can be a copolymer or a terpolymer of any of the monomers in any ratio. In an embodiment, the polybutene contains at least about 90% (wt) of isobutylene or 1-butene. In a particular embodiment, the polybutene contains at least about 90% (wt) of isobutylene. The polybutene may contain non-interfering amounts of other ingredients or additives, for instance it can contain up to 1000 ppm of an antioxidant (e.g., 2,6-di-tert-butyl-methylphenol). By way of example, the polybutene can have a molecular weight between about 150 kilodaltons and about 1,000 kilodaltons. In an embodiment, the polybutene can have between about 200 kilodaltons and about 600 kilodaltons. In a particular embodiment, the polybutene can have between about 350 kilodaltons and about 500 kilodaltons. Polybutenes having a molecular weight greater than about 600 kilodaltons, including greater than 1,000 kilodaltons are available but are expected to be more difficult to work with.

Additional alternative second polymers include diolefin-derived, non-aromatic polymers and copolymers, including those in which the diolefin monomer used to prepare the polymer or copolymer is selected from butadiene (CH₂═CH—CH═CH₂) and/or isoprene (CH₂═CH—C(CH₃)═CH₂). In an embodiment, the polymer is a homopolymer derived from diolefin monomers or is a copolymer of diolefin monomer with non-aromatic mono-olefin monomer, and optionally, the homopolymer or copolymer can be partially hydrogenated. Such polymers can be selected from the group consisting of polybutadienes prepared by the polymerization of cis-, trans- and/or 1,2-monomer units, or from a mixture of all three monomers, and polyisoprenes prepared by the polymerization of cis-1,4- and/or trans-1,4-monomer units. Alternatively, the polymer is a copolymer, including graft copolymers, and random copolymers based on a non-aromatic mono-olefin monomer such as acrylonitrile, and an alkyl(meth)acrylate and/or isobutylene. In an embodiment, when the mono-olefin monomer is acrylonitrile, the interpolymerized acrylonitrile is present at up to about 50% by weight; and when the mono-olefin monomer is isobutylene, the diolefin is isoprene (e.g., to form what is commercially known as a “butyl rubber”). Exemplary polymers and copolymers have a molecular weight between about 150 kilodaltons and about 1,000 kilodaltons. In an embodiment, polymers and copolymers have a molecular weight between about 200 kilodaltons and about 600 kilodaltons.

Additional alternative second polymers include aromatic group-containing copolymers, including random copolymers, block copolymers and graft copolymers. In an embodiment, the aromatic group is incorporated into the copolymer via the polymerization of styrene. In a particular embodiment, the random copolymer is a copolymer derived from copolymerization of styrene monomer and one or more monomers selected from butadiene, isoprene, acrylonitrile, a C₁-C₄ alkyl(meth)acrylate

(e.g., methyl methacrylate) and/or butene. Useful block copolymers include copolymer containing (a) blocks of polystyrene, (b) blocks of a polyolefin selected from polybutadiene, polyisoprene and/or polybutene (e.g., isobutylene), and (c) optionally a third monomer (e.g., ethylene) copolymerized in the polyolefin block. The aromatic group-containing copolymers contain about 10% to about 50% (wt.) of polymerized aromatic monomer and the molecular weight of the copolymer is from about 300 kilodaltons to about 500 kilodaltons. In an embodiment, the molecular weight of the copolymer is from about 100 kilodaltons to about 300 kilodaltons.

Additional alternative second polymers include epichlorohydrin homopolymers and poly(epichlorohydrin-co-alkylene oxide) copolymers. In an embodiment, in the case of the copolymer, the copolymerized alkylene oxide is ethylene oxide. By way of example, epichlorohydrin content of the epichlorohydrin-containing polymer is from about 30% to 100% (wt). In an embodiment, epichlorohydrin content is from about 50% to 100% (wt). In an embodiment, the epichlorohydrin-containing polymers have a molecular weight from about 100 kilodaltons to about 300 kilodaltons.

Non-degradable polymers can also include those described in U.S. Publ. Pat. App. No. 2007/0026037, entitled “DEVICES, ARTICLES, COATINGS, AND METHODS FOR CONTROLLED ACTIVE AGENT RELEASE OR HEMOCOMPATIBILITY”, the contents of which are herein incorporated by reference in its entirety. As a specific example, non-degradable polymers can include random copolymers of butyl methacrylate-co-acrylamido-methyl-propane sulfonate (BMA-AMPS). In some embodiments, the random copolymer can include AMPS in an amount equal to about 0.5 mol. % to about 40 mol. %.

Matrix forming polymers used with embodiments of the invention can also include polymers including one or more charged group. For example, matrix forming polymers of the invention can include polymers with positively charged groups and/or negatively charged groups.

### Substrates

In accordance with some embodiments herein, a coating including nucleic acids can be disposed on a substrate. Exemplary substrates can include metals, polymers, ceramics, and natural materials. Substrate polymers include those formed of synthetic polymers, including oligomers, homopolymers, and copolymers resulting from either addition or condensation polymerizations. Examples include, but not limited to, acrylics such as those polymerized from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; vinyls such as ethylene, propylene, styrene, vinyl chloride, vinyl acetate, vinyl pyrrolidone, and vinylidene difluoride, condensation polymers including, but are not limited to, polyamides such as polycaprolactam, polylauryl lactam, polyhexamethylene adipamide, and polyhexamethylene dodecanediamide, and also polyurethanes, polycarbonates, polysulfones, poly(ethylene terephthalate), polytetrafluoroethylene, polyethylene, polypropylene, polylactic acid, polyglycolic acid, polysiloxanes (silicones), cellulose, and polyetheretherketone.

Embodiments of the invention can also include the use of ceramics as a substrate. Ceramics include, but are not limited to, silicon nitride, silicon carbide, zirconia, and alumina, as well as glass, silica, and sapphire.

Substrate metals can include, but are not limited to, cobalt, chromium, nickel, titanium, tantalum, iridium, tungsten and alloys such as stainless steel, nitinol or cobalt chromium. Suitable metals can also include the noble metals such as gold, silver, copper, platinum, and alloys including the same.

Certain natural materials can also be used in some embodiments including human tissue, when used as a component of a device, such as bone, cartilage, skin and enamel; and other organic materials such as wood, cellulose, compressed carbon, rubber, silk, wool, and cotton. Substrates can also include carbon fiber. Substrates can also include resins, polysaccharides, silicon, or silica-based materials, glass, films, gels, and membranes.

However, it will be appreciated that embodiments of the invention can also be used without substrates. By way of example, embodiments can include a matrix with nucleic acid complexes disposed therein in the form of a filament or other shape without including a substrate.

### Further Methods

In an embodiment, the invention can include a method of making an implantable medical device. The method can include selecting a concentration of a cationic carrier agent corresponding to a desired elution profile. It will be appreciated that a desirable elution rate for an active agent can depend on various factors including the specific active agent used, the condition to be treated, etc. As shown below in the examples, devices in accordance with various embodiments herein can be made to have a desirable elution profile through modification of the components within the coating. In some embodiments, the release rate of the nucleic acid active agent can be related to the amount of the cationic carrier agent disposed within the coating. As such, a specific concentration of a cationic carrier agent can be selected that will lead to a specific elution profile.

Then, the method can also include combining a matrix forming polymer, a nucleic acid active agent, a solvent, and the cationic carrier agent to form a coating composition having the selected concentration of the cationic carrier agent, the active agent comprising nucleic acids. In some embodiments, the solvent used can be a non-aqueous solvent so as to prevent complexation between the nucleic acid active agent and the cationic carrier agent. The nucleic acid active agent can be processed in various ways prior to its incorporation into the coating composition. For example, the nucleic acid can be lyophilized, or otherwise converted into a particulate material.

Finally, the method can also include depositing the coating composition onto the surface of a substrate. It will be appreciated that there are many techniques for applying a coating onto a substrate. For example, the coating composition can be applied using dip coating, brush coating, printing processes, inkjet systems, spray coating, blade coating, and the like. However, while not intending to be bound by theory, spray coating is believed to be advantageous for various reasons including the ability to finely control the amount of the coating composition deposited.

It will be appreciated that embodiments of the invention can include and can be used with many different types of medical devices including implantable, and transitorily implantable, devices.

The present invention may be better understood with reference to the following examples. These examples are intended to be representative of specific embodiments of the invention, and are not intended as limiting the scope of the invention.

### EXAMPLES

### Example 1

### Formation of Coating with siRNA and PEI

100 μg Fluorescein-labeled siRNA (Operon/MWG Biotechnologies, Hunstville, Ala.) was precipitated by adding a 5 M NaCl solution and three times the volume of cold ethanol. The samples were then frozen at −20° C. for 30 minutes, thawed and spun at 10 krpm for 4 minutes. The pellets were washed once with 300 μl ethanol and spun again. Ethanol (supernatant) was completely removed and the pellets were fully dispersed in 300 μl chloroform, using a sonication bath. To the eye no particles could be seen and the solution was slightly orange.

The dispersion was then added to 4 ml chloroform containing polymers in different coating formulations shown in Table 1 below (for 100 μg of siRNA, a total of 2 mg polymers was used). The polymers used included polyethylene-co-vinyl acetate (“PEVA”), poly-n-butyl methacrylate (“PBMA”), a block copolymer of 80 wt. % polyethylene glycol (M.W.≈1000) and 20 wt. % polybutylene terephthalate (“1000PEG80PBT20”), a block copolymer of 55 wt. % polyethylene glycol (M.W.≈1000) and 45 wt. % polybutylene terephthalate (“1000PEG55PBT45”), polyvinylpyrrolidone (“PVP”), and polyethyleneimine branched 25 kDa (“PEI”) (Sigma, St. Louis, Mo.).

TABLE 1

| Formulation | PEVA | PBMA | 1000PEG80PBT20 | 1000PEG55PBT45 | PVP | PEI | siRNA |
|---|---|---|---|---|---|---|---|
| A | 20 | 20 | 55 | 0 | 0 | 0 | 5 |
| B | 20 | 20 | 0 | 45 | 0 | 10 | 5 |
| C | 25 | 25 | 0 | 50 | 0 | 0 | 0 |
| D | 20 | 20 | 0 | 45 | 10 | 0 | 5 |
| E | 20 | 20 | 0 | 55 | 0 | 0 | 5 |

Pieces of thin aluminum foil of approximately 3×3 cm were weighed and then coated with the various formulations. A spray coating apparatus with an ultrasonic spray head was used to apply the formulations onto the separate pieces of aluminum foil. For the coating process, the flow speed of the coating solution through the ultrasonic spray head was set to 0.07 ml/min, nitrogen gas pressure was set at 2.8 psi, and the power setting was at 0.8 Watts.

The coated pieces were then weighed again in order to determine coating weight and then the thin foil pieces were cut in four. Three of the four pieces were put in 1 ml of PBS at 37° C. to determine the release rate. One piece was used for surface characterization using both light and electron microscopy.

Observations with both light and electron microscopy established that all formulations resulted in stable coatings containing evenly distributed siRNA particulates.

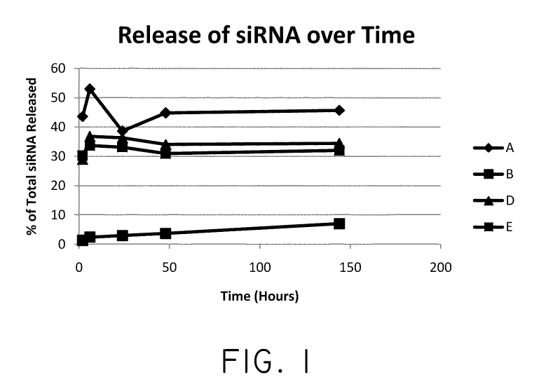

The elution results are shown below in Table 2 and in FIG. 1.

TABLE 2

| | Time (Hours) | | | | |
|---|---|---|---|---|---|
| Formulation | 2 | 6 | 24 | 48 | 144 |
| A | 43.592 | 52.946 | 38.623 | 44.794 | 45.695 |
| B | 1.325 | 2.427 | 2.945 | 3.675 | 6.990 |
| D | 28.951 | 36.799 | 36.333 | 34.023 | 34.438 |
| E | 30.255 | 33.652 | 33.151 | 30.928 | 31.962 |

This example shows that by adding PEI to the coating solution, near-linear control of siRNA release was obtained (17% of coated siRNA released in 14 days). Without PEI, using PEVA/PBMA/PEG-PBT, with or without PVP, a burst release was obtained without any further controlled release.

The integrity of released siRNA was then verified using electrophoresis through a polyacrylamide gel (15%). It was concluded that all of the siRNA was intact as it ran the same as the control siRNA.

### Example 2

### Effect of Varying Amounts of PEI on siRNA Elution

Fluorescently-labeled siRNA (100 μg) was precipitated by adding 5 M NaCl solution and three times the volume of cold ethanol. The samples were then frozen at −20° C. for 30 minutes, thawed and spun at 10 krpm for 4 minutes. The pellets were washed once with 300 μl ethanol and spun again. Ethanol was completely removed and the pellets were fully dispersed in 300 μl chloroform, using a sonication bath. To the eye no particles could be seen and the solution was slightly orange.

The dispersion was then added to different coating formulations each with 100 μg siRNA (5% w/w) and a total polymer weight of 2 mg (95% w/w) in a total of 4 ml chloroform. In all formulations the ratio between PEVA, PBMA and 1000PEG55PBT45 was 4:4:11. Different percentages of PEI (branched, 25 kDa) was then added to the formulation in amounts ranging from 1, 5, 10 to 25% w/w of the total formulation. The final percentages by weight (solids) of the different formulations are shown below in Table 3.

TABLE 3

| Formulation | PEVA | PBMA | 1000PEG55PBT45 | PEI | siRNA |
|---|---|---|---|---|---|
| F | 19.8 | 19.8 | 54.45 | 1 | 4.95 |
| G | 19.0 | 19.0 | 52.25 | 5 | 4.75 |
| H | 18.0 | 18.0 | 49.50 | 10 | 4.50 |
| I | 15.0 | 15.0 | 41.25 | 25 | 3.75 |

Pieces of thin aluminum foil of approximately 3×3 cm were weighed and then coated with the various formulations. A spray coating apparatus with an ultrasonic spray head was used to apply the formulations onto the separate pieces of aluminum foil. For the coating process, the flow speed of the coating solution through the ultrasonic spray head was set to 0.07 ml/min, nitrogen gas pressure was set at 2.8 psi, and the power setting was at 0.8 Watts.

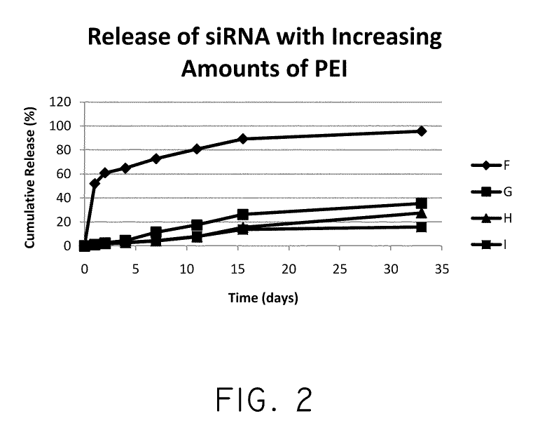

The coating weight was determined and the thin foil pieces were cut in four. The pieces were put in 1 ml of 10 mM (4-(2-hydroxyethyl)-1-piperazineethanesulfonic acid) (HEPES) buffer at 37° C. to determine the controlled release. The elution results are shown below in Table 4 and in FIG. 2. This example shows that the elution rate of the siRNA can be manipulated by varying the amount of PEI in the coating.

TABLE 4

| Time | Formulation | | | | | | | |
|---|---|---|---|---|---|---|---|---|
| (days) | F | F (SD) | G | G (SD) | H | H (SD) | I | I (SD) |
| 0 | 0 | 0 | 0 | 0 | 0 | 0 | 0 | 0 |
| 1 | 51.9419 | 14.70241 | 1.070582 | 0.039566 | 1.071977 | 0.184589 | 1.626699 | 0.175736 |
| 2 | 60.91283 | 15.8973 | 2.574596 | 0.165871 | 1.850965 | 0.350029 | 2.275809 | 0.193172 |
| 4 | 64.73413 | 16.52195 | 4.483852 | 0.320522 | 2.687083 | 0.542921 | 2.940675 | 0.203068 |
| 7 | 72.72552 | 20.00111 | 11.40584 | 3.174701 | 4.186464 | 0.834024 | 4.294225 | 0.154145 |
| 11 | 80.71334 | 21.85213 | 17.52622 | 4.397226 | 7.5158 | 1.129045 | 7.981858 | 0.221706 |
| 15.5 | 89.2646 | 23.1044 | 26.16709 | 3.730142 | 15.37206 | 2.568857 | 13.60176 | 0.235474 |
| 33 | 95.61325 | 24.22808 | 35.46915 | 2.658112 | 27.49111 | 3.723065 | 15.69719 | 1.245848 |

### Example 3

### Effects of Varying Amounts of PEI on siRNA Elution

Fluorescently-labeled siRNA (100 μg) was precipitated by adding 5 M NaCl solution and three times the volume of cold ethanol. The samples were then frozen at −20° C. for 30 minutes, thawed and spun at 10 krpm for 4 minutes. The pellets were washed once with 300 μl ethanol and spun again. Ethanol was completely removed and the pellets were fully dispersed in 300 μl chloroform, using a sonication bath. To the eye no particles could be seen and the solution was slightly orange.

The dispersion was then added to different coating formulations each with 100 μg siRNA (5% w/w) and a total polymer weight of 2 mg (95% w/w) in a total of 4 ml chloroform. In all formulations the ratio between PEVA, PBMA and 1000PEG55PBT45 was 1:1:2. Different percentages of PEI (branched, 25 kDa) were added to the formulation in amounts of 1, 2, 3, 4, 5 and 10% w/w of the total formulation. The final percentages by weight (solids) of the different formulations are shown below in Table 5.

TABLE 5

| Formulation | PEVA | PBMA | 1000PEG55PBT45 | PEI | siRNA |
|---|---|---|---|---|---|
| J | 23.51 | 23.51 | 47.03 | 1 | 4.95 |
| K | 23.28 | 23.28 | 46.55 | 2 | 4.90 |
| L | 23.04 | 23.04 | 46.08 | 3 | 4.85 |
| M | 22.80 | 22.80 | 45.60 | 4 | 4.80 |
| N | 22.56 | 22.56 | 45.13 | 5 | 4.75 |
| O | 21.38 | 21.38 | 42.75 | 10 | 4.50 |

Pieces of thin aluminum foil of approximately 3×3 cm were weighed and then coated with the various formulations. A spray coating apparatus with an ultrasonic spray head was used to apply the formulations onto the separate pieces of aluminum foil. For the coating process, the flow speed of the coating solution through the ultrasonic spray head was set to 0.07 ml/min, nitrogen gas pressure was set at 2.8 psi, and the power setting was at 0.8 Watts.

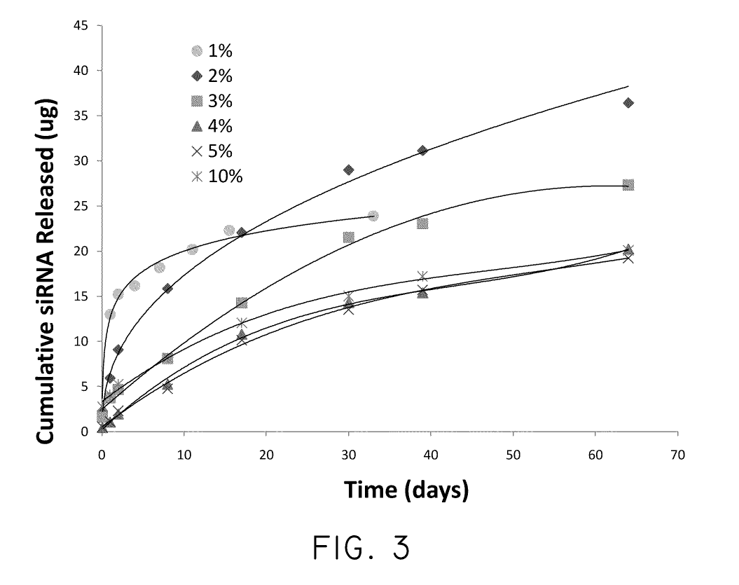

The coating weight was determined and the thin foil pieces were cut in four. The pieces were put in 1 ml of 10 mM HEPES buffer at 37° C. to determine the controlled release. The elution results are shown below in Tables 6 & 7 and in FIG. 3. This example also shows that the elution rate of the siRNA can be manipulated by varying the amount of PEI in the coating.

TABLE 6

| Time | Cumulative Release | | | | |
|---|---|---|---|---|---|
| (days) | 2% PEI | 3% PEI | 4% PEI | 5% PEI | 10% PEI |
| 0.083 | 2.301854 | 1.627899 | 0.453944 | 0.527584 | 2.789435 |
| 1 | 5.93194 | 3.763232 | 1.085954 | 1.114203 | 4.092251 |
| 2 | 9.083628 | 4.698111 | 1.962148 | 2.313031 | 5.26939 |
| 8 | 15.854 | 8.094245 | 5.274147 | 4.771698 | 8.18975 |
| 17 | 22.06236 | 14.29054 | 10.825 | 10.16337 | 12.06419 |

TABLE 7

| | Cumulative |
|---|---|
| Time | Release |
| (days) | 1% PEI |
| 0 | 0 |
| 1 | 12.98548 |
| 2 | 15.22821 |
| 4 | 16.18353 |
| 7 | 18.18138 |
| 11 | 20.17834 |
| 15.5 | 22.31615 |

### Example 4

### Formation of Coating with DNA/Dextran Particles and PEI

Plasmid DNA encoding for luciferase (Aldevron, Fargo, N. Dak.) was co-phase separated in polyethylene glycol (PEG) with dextran. Specifically, 100 μl DNA 1 μg/μl was added to 50 μl dextran (Sigma, 35-45 kDa) solution 100 mg/ml in distilled deionized water (DDW). 500 μl of a 30% w/w PEG 20 kDa in DDW solution was then added. The resulting turbid mixture was put on dry ice and lyophilized.

The PEG was then removed from the lyophilized cake by adding chloroform followed by centrifugation and subsequent removal of the chloroform-PEG phase. This washing procedure was done twice. The resulting dextran-DNA particles were then re-suspended in chloroform.

Different formulations of dextran-DNA particles with other components were then prepared. The following solutions were prepared:

Solution 1: PEVA/PBMA/1000PEG55PBT45 1:1:2 ratio, 40 mg/ml in chloroform

Solution 2: polyethyleneimine (PEI) 25 kDa 10 mg/ml in chloroform

Solution 3: 5000PEG-PEI 10 mg/ml in methanol/chloroform 1:1

The 5000PEG-PEI solution was prepared using carbonyldiimidazole (CDI) based chemistry. 5000 Da methycapped polyethylene glycol (mPEG) was activated by CDI to generate mPEG-imidazole. PEI was dissolved in dichloromethane at a 4 mM concentration. mPEG-imidazole was added to the PEI solution at a 3 fold molar excess. The solution was stirred for 1 hour and then dried overnight in a vacuum oven to generate 5000PEG-PEI.

These solutions were used to create formulations based on 40% w/w particles (20 mg), 60% polymer (30 mg). For formulation the solutions were mixed together, along with the dextran-DNA particles, and then sprayed onto the substrate with a single spray head. For example, for formulation P, solution 1 was mixed with solution 2 along with the particles. For formulation Q, solution 1 was mixed with solution 3 along with the particles. The final percentages by weight (solids) of the different formulations are shown below in Table 8.

TABLE 8

| | DNA/Dextran | | | | | |
|---|---|---|---|---|---|---|
| Formulation | Particles | PEVA | PBMA | 1000PEG55PBT45 | PEI | PEG/PEI |
| P | 40% | 13.75% | 13.75% | 27.5% | 5% | 0% |
| Q | 40% | 13.75% | 13.75% | 27.5% | 0% | 5% |
| R | 40% | 15% | 15% | 30% | 0% | 0% |
| S | 40% | 15% | 15% | 30% | 0% | 0% |

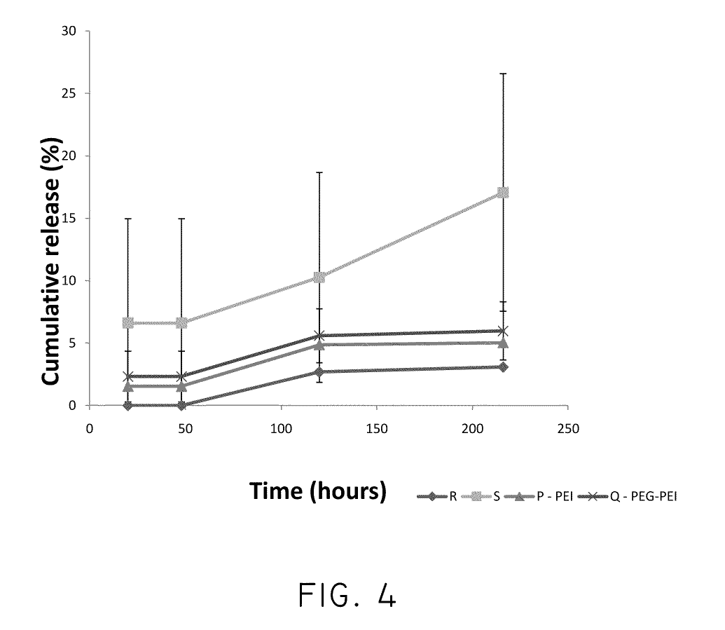

On a sheet of thin aluminum foil areas of about 3×4 cm were coated with a 20 mg/ml solution of PEVA/PBMA 1:1 in chloroform (three passes total) using a spray coating apparatus with an ultrasonic spray head (flow speed 0.07 ml/min, nitrogen gas pressure at 2.8 psi, and the power setting at 0.8 Watts). Then a given formulation (P, Q, R, or S) was coated on top of the base-coat at the same rate and settings in a total amount of 5 mg. For formulation S, a topcoat of solution 1 was then applied in amount of approximately 12 mg. The thin foil pieces were cut in four and were put in 1 ml of 10 mM HEPES buffer at 37° C. to determine the controlled release. The elution results are shown in FIG. 4. It was found that by 220 hours a small amount (˜2-15% of loading) of DNA was released from all formulations.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration to. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference. Nothing herein is to be construed as an admission that the inventors are not entitled to antedate any publication and/or patent, including any publication and/or patent cited herein.

The invention has been described with reference to various specific and preferred embodiments and techniques. However, it should be understood that many variations and modifications may be made while remaining within the spirit and scope of the invention.

### Further Embodiments

In a embodiment the invention includes an active agent eluting coating including a polymeric matrix, a cationic carrier agent disposed with the matrix, and an active agent disposed within the matrix, the active agent comprising nucleic acids substantially uncomplexed with the cationic carrier agent. In an embodiment, the cationic carrier agent includes polyethyleneimine. In an embodiment, the cationic carrier agent is between about 0.1% and 25% by weight of the coating. In an embodiment, the cationic carrier agent comprising between about 1% and 10% by weight of the coating. In an embodiment, the polymeric matrix includes polymers soluble in non-aqueous solvents. In an embodiment, the polymeric matrix includes polymers having a solubility parameter of less than about 11.0 (cal/cm3)1/2. In an embodiment, the polymeric matrix includes degradable and non-degradable polymers. In an embodiment, the polymeric matrix includes polyethylene-co-vinyl acetate and poly-n-butyl methacrylate and a block copolymer of polyethylene glycol and polybutylene terephthalate. In an embodiment, the nucleic acids are selected from the group consisting of ribonucleic acids (RNA), deoxyribonucleic acids (DNA), small interfering RNA (siRNA), micro RNA (miRNA), piwi-interacting RNA (piRNA), short hairpin RNA (shRNA), antisense nucleic acids, aptamers, ribozymes, locked nucleic acids, and catalytic DNA. In an embodiment, at least about 95 percent of the nucleic acids are uncomplexed with the cationic carrier agent. In an embodiment, the active agent is configured to elute out of the polymeric matrix when the coating is disposed in an aqueous solvent. In an embodiment, the active agent is configured to form complexes with cationic carrier agent when the coating is disposed in an aqueous solvent. In an embodiment, the coating is configured to elute complexes of the cationic carrier agent and the active agent, the complexes capable of transfecting target cells. In an embodiment, the coating includes at least about 5 percent by weight of nucleic acids.

In an embodiment, the invention includes an implantable medical device including a substrate, and a coating disposed on the substrate. The coating can include a polymeric matrix, a cationic carrier agent disposed with the matrix, and an active agent disposed within the matrix, the active agent including nucleic acids substantially uncomplexed with the cationic carrier agent.

In an embodiment, the invention includes a method of making an implantable medical device. The method can include selecting a concentration of a cationic carrier agent corresponding to a desired elution profile; combining a matrix forming polymer, an active agent, a solvent, and the cationic carrier agent to form a coating composition having the selected concentration of the cationic carrier agent, the active agent comprising nucleic acids; and depositing the coating composition onto the surface of a substrate.

In an embodiment, the invention includes a liquid composition for forming an active agent eluting coating, the liquid composition including a matrix forming polymer; a cationic carrier agent; an active agent, the active agent comprising nucleic acids; and a solvent, wherein the active agent is not soluble in the solvent. In an embodiment, the solvent includes a non-aqueous solvent. In an embodiment the liquid composition is suitable to be sprayed onto a substrate.

In an embodiment, the invention includes a method of making an implantable medical device. The method can include combining a matrix forming polymer mixture, a cationic carrier agent, and an active agent together with a non-aqueous solvent to form a monophasic coating composition, the active agent comprising nucleic acids; and depositing the coating solution onto the surface of a substrate. In an embodiment, depositing the coating solution onto the surface of a substrate comprises spraying the coating solution.

The claims are:

1. An active agent eluting coating comprising:
a polymeric matrix,
a cationic carrier agent disposed with the matrix, and
an active agent disposed within the matrix, the active agent comprising nucleic acids substantially uncomplexed with the cationic carrier agent.

2. The active agent eluting coating of claim 1, the cationic carrier agent soluble in a non-aqueous solvent.

3. The active agent eluting coating of claim 1, the cationic carrier agent comprising polyethyleneimine.

4. The active agent eluting coating of claim 1, the cationic carrier agent comprising between about 0.1% and 25% by weight of the coating.

5. The active agent eluting coating of claim 1, the cationic carrier agent comprising between about 1% and 10% by weight of the coating.

6. The active agent eluting coating of claim 1, the polymeric matrix comprising polymers soluble in non-aqueous solvents.

7. The active agent eluting coating of claim 1, the polymeric matrix comprising polymers having a solubility parameter of less than about 11.0 (cal/cm3)1/2.

8. The active agent eluting coating of claim 1, the polymeric matrix comprising degradable and non-degradable polymers.

9. The active agent eluting coating of claim 1, the polymeric matrix comprising polyethylene-co-vinyl acetate and poly-n-butyl methacrylate and a degradable block copolymer comprising amphiphilic blocks.

10. The active agent eluting coating of claim 1, the nucleic acids selected from the group consisting of ribonucleic acids (RNA), deoxyribonucleic acids (DNA), small interfering RNA (siRNA), micro RNA (miRNA), piwi-interacting RNA (piRNA), short hairpin RNA (shRNA), antisense nucleic acids, aptamers, ribozymes, locked nucleic acids, catalytic DNA, and derivatives thereof.

11. The active agent eluting coating of claim 1, wherein at least about 95 percent of the nucleic acids are uncomplexed with the cationic carrier agent.

12. The active agent eluting coating of claim 1, the active agent configured to elute out of the polymeric matrix when the coating is disposed in an aqueous solvent.

13. The active agent eluting coating of claim 1, the active agent configured to form complexes with the cationic carrier agent when the coating is disposed in an aqueous solvent, the complexes capable of transfecting target cells.

14. The active agent eluting coating of claim 1, the coating comprising at least about 0.1 percent by weight of nucleic acids.

15. An implantable medical device comprising:
a substrate, and
a coating disposed on the substrate, the coating comprising
a polymeric matrix,
a cationic carrier agent disposed with the matrix, and
an active agent disposed within the matrix, the active agent comprising nucleic acids substantially uncomplexed with the cationic carrier agent.

16. The implantable medical device of claim 15, the cationic carrier agent soluble in a non-aqueous solvent.

17. The implantable medical device of claim 15, the cationic carrier agent comprising polyethyleneimine.

18. The active agent eluting coating of claim 15, the active agent configured to form complexes with the cationic carrier agent when the coating is disposed in an aqueous solvent, the complexes capable of transfecting target cells.

19. A method of making an implantable medical device comprising:
selecting a concentration of a cationic carrier agent corresponding to a desired elution profile;
combining a matrix forming polymer, an active agent, a solvent, and the cationic carrier agent to form a coating composition having the selected concentration of the cationic carrier agent, the active agent comprising nucleic acids; and
depositing the coating composition onto the surface of a substrate.

20. The method of making an implantable medical device of claim 19, the cationic carrier agent comprising polyethyleneimine.

21. The method of making an implantable medical device of claim 19, wherein the concentration of cationic carrier agent selected is greater than about 1.0 wt. %.
